(19) United States
(12) Patent Application Publication
(10) Pub. No.: US 2024/0074948 A1
(43) Pub. Date: Mar. 7, 2024

(54) STICK-SHAPED COSMETIC MATERIAL

(71) Applicant: TOKIWA CORPORATION, Nakatsugawa-shi (JP)

(72) Inventors: Yoshinori AKAMATSU, Kawaguchi-shi (JP); Natsuko MOMOKITA, Kawaguchi-shi (JP)

(21) Appl. No.: 18/460,285

(22) Filed: Sep. 1, 2023

(30) Foreign Application Priority Data
Sep. 7, 2022 (JP) 2022-142285

(51) Int. Cl. A61K 8/02; A61K 8/37; A61K 8/92; A61Q 1/02

(52) U.S. Cl. CPC A61K 8/0233; A61K 8/375; A61K 8/92; A61Q 1/02

(57) ABSTRACT

A stick-shaped cosmetic material including two or more regions divided in a longitudinal direction of the stick-shaped cosmetic material, in which the two or more regions include a first region containing a first cosmetic composition containing a wax (A1) having a melting point of 65° C. or higher as an oil component in the first cosmetic composition, and a second region containing a second cosmetic composition containing, as oil components in the second cosmetic composition, the wax (A1) and a fatty acid triglyceride (A2) having a melting point of 30° C. or higher, the first region and the second region are adjacent to each other, and when content ratios (mass %) of the wax (A1) based on a total amount of the oil components in the first cosmetic composition and the second cosmetic composition are defined as M1 and M2, respectively, M1>M2 is satisfied.

### CROSS-REFERENCE TO RELATED APPLICATIONS

This application is based on and claims priority under 35 USC § 119 from Japanese Patent Application No. 2022-142285 filed on Sep. 7, 2022, the contents of which are incorporated herein by reference.

### TECHNICAL FIELD

The present invention relates to a stick-shaped cosmetic material.

### BACKGROUND ART

A stick-shaped cosmetic material for makeup such as an eyebrow pencil, a lipstick, a lip liner, a pencil eyeliner, and a concealer contains an oil component such as a solid oil and a powder component such as a coloring material. In order to perform desired makeup, two or more cosmetic materials having different colors are used in combination, and from a viewpoint of portability and operability, a pencil-like cosmetic material in which two or more layers having different colors are separately formed has been proposed (for example, see Patent Literature 1 below).

### CITATION LIST

### Patent Literature Patent Literature 1: JP2010-30969A

### SUMMARY OF INVENTION

### Technical Problem

In makeup, items having different makeup effects may be selectively used for the same region. For example, in makeup of eyebrows, an eyebrow pencil and an eyebrow powder are used in combination. The eyebrow pencil contains an oil component and a powder component such as a pigment, and can draw an outline of the whole eyebrow or can create strokes of eyebrows. On the other hand, the eyebrow powder contains a large amount of extender pigments such as mica, talc, and silica, and thus the eyebrow powder can achieve a makeup effect with a soft and feathered finish on the whole eyebrow. The present inventor focused attention on a combination of these items, and found out the following problems as a result of a study on a stick-shaped cosmetic material that allows for selectively drawing with different makeup effects.

That is, the prevent inventors found that integrating layers having different hardness in one cosmetic material in order to selectively draw with different makeup effects may occur some problems such as difficulty of drawing a uniform line due to catching, large unbalanced reduction of a layer having small hardness because of early reduction of the layer having small hardness, and occurrence of chipping due to detachment of a part of the layer. In a related stick-shaped cosmetic material aiming at selectively drawing a plurality of colors as in a core material described in Patent Literature 1, configurations of oil components in each layer are similar, and this makes it difficult to obtain a difference in makeup effects. The study executed by the present inventors has revealed that even integrating layers having different hardness may not allow to selectively draw with different makeup effects.

The present invention is made in view of the above circumstances, and an object of the present invention is to provide a stick-shaped cosmetic material that allows for selectively drawing with different makeup effects and achieves to sufficiently prevent catching, unbalanced reduction, and chipping in use.

### Solution to Problem

In order to solve the above problems, the following inventions are provided.
1) A stick-shaped cosmetic material including two or more regions divided in a longitudinal direction of the stick-shaped cosmetic material, in which the two or more regions include a first region including a first cosmetic composition containing a wax (A1) having a melting point of 65° C. or higher as an oil component in the first cosmetic composition, and a second region including a second cosmetic composition containing, as oil components in the second cosmetic composition, the wax (A1) having a melting point of 65° C. or higher and a fatty acid triglyceride (A2) having a melting point of 30° C. or higher, the first region and the second region are adjacent to each other, and when a content ratio (mass %) of the wax (A1) based on a total amount of the oil components in the first cosmetic composition and a content ratio (mass %) of the wax (A1) based on a total amount of the oil components in the second cosmetic composition are defined as M1 and M2, respectively, M1>M2 is satisfied.
2) The stick-shaped cosmetic material according to 1), in which, when hardness (N) at 25° C. of the first cosmetic composition and hardness (N) at 25° C. of the second cosmetic composition are defined as H1 and H2, respectively, H1>H2 is satisfied.
3) The stick-shaped cosmetic material according to 1) or 2), in which the first cosmetic composition contains hydrogenated castor oil as the wax (A1).
4) The stick-shaped cosmetic material according to any one of 1) to 3), in which the first cosmetic composition contains the fatty acid triglyceride (A2) having a melting point of 30° C. or higher.
5) The stick-shaped cosmetic material according to 4), in which a content of the wax (A1) and a content of the fatty acid triglyceride (A2) in the first cosmetic composition are respectively 30 mass % to 37 mass % and 3 mass % to 7 mass % based on the amount of the first cosmetic composition, a content of the wax (A1) and a content of the fatty acid triglyceride (A2) in the second cosmetic composition are respectively 20 mass % to 30 mass % and 2 mass % to 13 mass % based on the amount of the second cosmetic composition, and hardness at 25° C. of the first cosmetic composition and hardness at 25° C. of the second cosmetic composition are respectively 2.5 N to 4.5 N and 0.5 N to 2.5 N.

### Advantageous Effects of Invention

The present invention allows for providing a stick-shaped cosmetic material that allows for selectively drawing with different makeup effects and achieves to sufficiently prevent catching, unbalanced reduction, and chipping in use.

The stick-shaped cosmetic material of the present invention allows for imparting a clear drawn line and a feathered finish with one stick-shaped cosmetic material.

### BRIEF DESCRIPTION OF DRAWINGS

FIG. 1 is a perspective view illustrating an embodiment of a stick-shaped cosmetic material according to the present invention.

(a) to (c) of FIG. 2 illustrate schematic sectional views of other embodiments of the stick-shaped cosmetic material according to the present invention.

### DESCRIPTION OF EMBODIMENTS

### Stick-Shaped Cosmetic Material

The stick-shaped cosmetic material according to the present embodiment contains an oil component (A) (hereinafter, sometimes referred to as a component (A)) and a powder component (B) (hereinafter, sometimes referred to as a component (B)), and includes two or more regions divided in a longitudinal direction of the stick-shaped cosmetic material, and includes at least the following first and second regions as the two or more regions. The first region includes a first cosmetic composition containing a wax (A1) having a melting point of 65° C. or higher (hereinafter, sometimes referred to as a component (A1)). The second region includes a second cosmetic composition containing the wax (A1) having a melting point of 65° C. or higher and a fatty acid triglyceride (A2) having a melting point of 30° C. or higher (hereinafter, sometimes referred to as a component (A2)). The first region and the second region are adjacent to each other, and when content ratios (mass %) of the component (A1) based on a total amount of the oil component in the first cosmetic composition and the second cosmetic composition are M1 and M2, respectively, a relation of M1>M2 is satisfied. The first region and the second region may be partially in contact with each other.

The stick-shaped cosmetic material of the present disclosure has the above configuration, and thus, this allows for selectively drawing with different makeup effects, and sufficiently preventing catching, unbalanced reduction and chipping in use. That is, even decreasing hardness of the second region by a blending difference of the component (A1) (softening a makeup effect) achieves an appropriate sliding property due to the component (A2) contained in the second region. Thus, this would sufficiently prevent occurrence of problems such as difficulty of drawing a uniform line due to catching, large unbalanced reduction of a layer having small hardness because of early reduction of the layer having small hardness, and occurrence of chipping due to detachment of a part of the layer.

### Oil Component (A)

Typically, as the component (A), a solid oil, a paste oil, a liquid oil, or the like can be used with no limitation as long as it is an oil agent used for a cosmetic material. The oil component may be used alone or in combination of two or more thereof.

Examples of the component (A) include hydrocarbons such as paraffin wax, microcrystalline wax, polyethylene wax, synthetic wax, ceresin, vaseline, squalane, olefin oligomer, hydrogenated polyisobutene, light liquid isoparaffin, heavy liquid isoparaffin, and isododecane; vegetable oils and fats such as hydrogenated castor oil (hardened castor oil), hydrogenated jojoba oil, carnauba wax, rice bran wax, candelilla wax, and sunflower wax; vegetable oils such as sunflower seed oil, jojoba seed oil, olive oil, and castor oil; esters such as glyceryl tribehenate, dipentaerythrityl hexahydroxystearate/hexastearate/hexarosinate, dipentaerythrityl tetrahydroxystearate/tetraisostearate, dipentaerythrityl pentaisostearate, dipentaerythrityl hexahydroxystearate, hydrogenated castor oil isostearate, phytosteryl oleate, sucrose hexaoleate/hexapalmitate/hexastearate, phytosteryl macadamiate, cholesteryl hydroxystearate, cholesteryl oleate, bis-diglyceryl polyacyladipate-2, cetyl ethylhexanoate, ethylhexyl palmitate, triethylhexanoin, isotridecyl isononanoate, isostearyl isostearate, neopentyl glycol diethylhexanoate, caprylic/capric triglyceride, caprylic/capric/myristic/stearic triglyceride, neopentyl glycol dicaprate, propanediol diisostearate, trimethylolpropane triethylhexanoate, octyldodecyl myristate, octyldodecyl stearoyl stearate, diisostearyl malate, polyglyceryl triisostearate, trimethylolpropane triisostearate, sorbitan sesquiisostearate, sorbitan isostearate, and sorbitan sesquioleate; a higher fatty acid such as stearic acid, palmitic acid, oleic acid, behenic acid, and lauric acid; higher alcohols such as stearyl alcohol, behenyl alcohol, isostearyl alcohol, octyldodecanol, and oleyl alcohol; silicones such as acrylic modified silicone, dimethicone, methyl trimethicone, and phenyl trimethicone; and sucrose fatty acid esters such as dextrin palmitate and stearoyl inulin. These may be used alone or in combination of two or more.

Examples of the wax (A1) having a melting point of 65° C. or higher include the above hydrocarbons such as paraffin wax, microcrystalline wax, polyethylene wax, synthetic wax, and ceresin, hydrogenated castor oil (hardened castor oil), carnauba wax, sunflower wax, candelilla wax, and beeswax. The component (A1) may be used alone or in combination of two or more thereof.

In this specification, a melting point of the oil component is measured by the following method. About 5 mg of a sample is weighed and placed in an aluminum sample pan. An aluminum cover is attached to the pan, and the pan is installed in a differential scanning calorimeter “DSC7020” (manufactured by Hitachi High-Tech Science Corporation, product name). By using an electric cooling unit “Polyscience” (manufactured by Hitachi High-Tech Science Corporation, product name), the sample and a reference sample are held at −10° C. for 1 min under a nitrogen gas flow rate of 30 mL/min to 35 mL/min, and then heated from 0° C. to 120° C. with a heating rate of 10° C./min, cooled from 120° C. to −10° C. with a cooling condition of −10° C./min, held at −10° C. for 5 min, and then heated again from −10° C. to 120° C. with a heating rate of 10° C./min, to determine a melting endothermic curve. A peak temperature of the melting endothermic curve during the second heating at this time is set as the melting point. If there are a plurality of the peak temperatures, a peak temperature having a highest melting temperature is set as the melting point.

Examples of the fatty acid triglyceride (A2) having a melting point of 30° C. or higher include C10-18 triglycerides, caprylic/capric/myristic/stearic triglyceride, glyceryl tribehenate, glyceryl trimyristate, glyceryl trilaurate, and glyceryl tribehenate/isostearate/eicosandioate. The component (A2) may be used alone or in combination of two or more thereof.

From a viewpoint of prevention of unbalanced reduction, ease of application, and a feathered makeup effect with a powder texture, the component (A2) may be at least one selected from caprylic/capric/myristic/stearic triglyceride, C10-18 triglycerides, and glyceryl trilaurate.

From a viewpoint of prevention of unbalanced reduction and ease of application, a hydrocarbon group derived from a fatty acid may have 6 to 20 carbon atoms or 8 to 18 carbon atoms in the component (A2).

The melting point of the component (A2) may be 60° C. or lower, and the melting point may be 45° C. or lower from a viewpoint of selectively drawing in terms of makeup effects and the ease of drawing.

In the stick-shaped cosmetic material according to the present embodiment, the component (A2) and a liquid oil (A3) having a viscosity at 25° C. of 50 mPa·s or lower (hereinafter, sometimes referred to as (A3)) may be used in combination. In the present specification, the viscosity at 25° C. of the liquid oil refers to a value obtained by measuring a sample at 25° C. by using a B-type viscometer at a rotation speed of 12 rpm with a BL adapter.

Examples of the component (A3) include esters such as triethylhexanoin, isopropyl palmitate, ethylhexyl palmitate, hexyldecyl ethylhexanoate, cetyl ethylhexanoate, isononyl isononanoate, isotridecyl isononanoate, isopropyl isostearate, isostearyl isostearate, neopentyl glycol diethylhexanoate, caprylic/capric triglyceride, neopentyl glycol dicaprate, propanediol diisostearate, trimethylolpropane triethylhexanoate, isopropyl myristate, and octyldodecyl myristate; and silicones such as dimethicone and phenyl trimethicone.

### Powder Component (B)

In general, the component (B) is not limited as long as it is a powder to be used for a cosmetic material, but examples thereof include a coloring pigment (hereinafter, sometimes referred to as a component (B1)) and an extender powder (hereinafter, sometimes referred to as a component (B2)). The shape of the powder is not limited, but may have a particle structure such as a spherical shape, a plate shape, a needle-like shape, an aerosol shape, a fine particle, a pigment grade particle diameter, a porous shape, or a non-porous shape.

Examples of the coloring pigment include inorganic coloring pigments such as red iron oxide, yellow iron oxide, black iron oxide, cobalt oxide, chromium oxide, ultramarine, dark blue, titanium oxide, and zinc oxide; organic coloring pigments such as permaton red, helindone pink CN, phthalocyanine blue, lithol rubine BCA, and titanium aluminium lake; white pigments such as zinc oxide, titanium oxide, and cerium oxide; pearl pigments such as mica titanium, fine particle titanium oxide coated mica titanium, barium sulfate coated mica titanium, fish scale foil, bismuth oxychloride, and aluminum flake; and natural pigments such as carmines and safflower.

Examples of the extender powder include extender pigments such as mica, synthetic mica, sericite, talc, kaolin, silicon carbide, barium sulfate, bentonite, smectite, aluminum oxide, silica, magnesium oxide, zirconium oxide, magnesium carbonate, calcium carbonate, chromium oxide, aluminum hydroxide, and magnesium hydroxide; ultraviolet scattering agents such as titanium oxide fine particles and zinc oxide fine particles; organic powders such as a nylon powder, a polymethyl methacrylate powder, a acrylonitrile-methacrylic acid copolymer powder, a vinylidene chloride-methacrylic acid copolymer powder, a polyethylene powder, a polystyrene powder, an organopolysiloxane elastomer powder, a polymethylsilsesquioxane powder, a urethane powder, a wool powder, a silk powder, a cellulose powder, and a N-acyl lysine powder; composite powders such as fine particle titanium oxide coated mica titanium, fine particle titanium oxide coated nylon, barium sulfate coated mica titanium, titanium oxide containing silica, and zinc oxide containing silica; and metal soaps such as magnesium stearate, zinc myristate, aluminum stearate, and calcium stearate.

As the powder component, a hydrophobic treating powder may be used from a viewpoint of color development and adhesion. Examples of the hydrophobic treating powder include higher fatty acids, metal soaps, fats and oils, waxes, silicone compounds, fluorine compounds, surfactants, and dextrin fatty acid esters.

The powder component may be used alone or in combination of two or more thereof.

In addition to the components described above, the stick-shaped cosmetic material according to the present embodiment may contain other components used for an ordinary cosmetic material, for example, a surfactant, a preservative, an antioxidant, a dye, a thickening agent, a pH adjusting agent, a fragrance, an ultraviolet absorbent, an ultraviolet scattering agent, a chelating agent, a quenching agent, and a moisturizing agent.

### First Cosmetic Composition and Second Cosmetic Composition

The first cosmetic composition and the second cosmetic composition may contain the oil component and the powder component described above, and may contain other necessary components.

Contents of the component (A) in the first cosmetic composition and the second cosmetic composition may be 10 mass % to 90 mass % and 10 mass % to 90 mass %, may be 30 mass % to 85 mass % and 30 mass % to 80 mass %, may be 50 mass % to 75 mass % and 40 mass % to 70 mass %, and may be 55 mass % to 70 mass % and 50 mass % to 65 mass %, respectively, based on a total amount of the compositions.

Contents of the component (A1) in the first cosmetic composition and the second cosmetic composition may be 10 mass % to 60 mass % and 0.5 mass % to 50 mass %, may be 20 mass % to 50 mass % and 10 mass % to 40 mass %, and may be 30 mass % to 40 mass % and 20 mass % to 30 mass %, respectively, based on the total amount of the compositions.

Based on the total amount of the compositions, contents of the component (B) in the first cosmetic composition and the second cosmetic composition may be 10 mass % to 90 mass % and 10 mass % to 90 mass %, may be 20 mass % to 70 mass % and 20 mass % to 70 mass %, may be 35 mass % to 50 mass % and 30 mass % to 60 mass %, and may be 30 mass % to 45 mass % and 35 mass % to 50 mass % from a viewpoint of coloring effect and the ease of application, respectively.

In the stick-shaped cosmetic material according to the present embodiment, when the content ratios (mass %) of the component (A1) based on the total amount of the oil component in the first cosmetic composition and the second cosmetic composition are M1 and M2, respectively, the relation of M1>M2 is satisfied, but from the viewpoint of selectively drawing in terms of makeup effects, M1 and M2 may be 35 mass % to 70 mass % and 5 mass % to 60 mass %, may be 40 mass % to 65 mass % and 20 mass % to 55 mass %, and may be 45 mass % to 58 mass % and 40 mass % to 50 mass %, respectively.

The first cosmetic composition may contain a hydrogenated castor oil as the component (A1) from the viewpoint of selectively drawing with different makeup effects. In this case, a content of the hydrogenated castor oil may be 5 mass % to 30 mass % or 10 mass % to 20 mass % based on a total amount of the component (A) in the first cosmetic composition.

The first cosmetic composition may contain the component (A2) from the viewpoint of ease of application and prevention of unbalanced reduction. In this case, a content of the component (A2) may be 0.5 mass % to 15 mass % or 4 mass % to 10 mass % based on the total amount of the component (A) in the first cosmetic composition.

The first cosmetic composition may contain the component (A3) from a viewpoint of ease of application and ease of molding. In this case, a content of the component (A3) may be 15 mass % to 45 mass % or 20 mass % to 32 mass % based on the total amount of the component (A) in the first cosmetic composition.

The first cosmetic composition may contain the components (A2) and (A3) from the viewpoint of ease of application and ease of molding. In this case, a total content of the component (A2) and the component (A3) may be 20 mass % to 60 mass % or 25 mass % to 45 mass % based on the total amount of the component (A) in the first cosmetic composition from the viewpoint of ease of application. From the viewpoint of ease of application, the content of the component (A2) may be 5 parts by mass to 55 parts by mass or 15 parts by mass to 50 parts by mass with respect to 100 parts by mass of a total of the component (A2) and the component (A3).

The first cosmetic composition may contain the extender powder (B2) from a viewpoint of ease of application and finish with a powder texture. In this case, a content of the component (B2) may be 5 mass % to 30 mass % or 10 mass % to 20 mass % based on a total amount of the component (B) in the first cosmetic composition. Further, the component (B2) may be a plate-like powder, and may be one or more of mica, synthetic mica, sericite, and talc.

Based on a total amount of the component (A) in the second cosmetic composition, a content of the component (A2) in the second cosmetic composition may be 0.5 mass % to 35 mass % from the viewpoint of ease of application and selectively drawing with different makeup effects, may be 5 mass % to 30 mass % from the viewpoint of ease of drawing, and may be 12 mass % to 22 mass % from a viewpoint of achieving both the ease of drawing and the prevention of unbalanced reduction at a high level.

The second cosmetic composition may contain the components (A2) and (A3) from the viewpoint of ease of application. In this case, a total content of the component (A2) and the component (A3) may be 20 mass % to 70 mass % or 30 mass % to 60 mass % based on the total amount of the component (A) in the second cosmetic composition from the viewpoint of ease of application. From the viewpoint of achieving both the ease of application and the prevention of unbalanced reduction, the content of the component (A2) may be 10 parts by mass to 60 parts by mass or 15 parts by mass to 40 parts by mass with respect to 100 parts by mass of a total of the component (A2) and the component (A3).

The second cosmetic composition may contain the extender powder (B2) from the viewpoint of ease of application and selectively drawing with different makeup effects. In this case, a content of the component (B2) may be 30 mass % to 70 mass %, may be 35 mass % to 60 mass %, and may be 40 mass % to 55 mass % based on a total amount of the component (B) in the second cosmetic composition. Further, the component (B2) may be a plate-like powder, and may be one or more of mica, synthetic mica, sericite, and talc.

The stick-shaped cosmetic material according to the present embodiment may satisfy a relation of H1>H2 when hardness (N) at room temperature (25° C.) (hereinafter, also referred to as bulk hardness) of the first cosmetic composition and the second cosmetic composition are defined as H1 and H2, respectively. In this case, the first region functions as a hard layer and the second region functions as a soft layer, and different makeup effects can be easily obtained.

In the present specification, the bulk hardness of the cosmetic composition is determined by the following measurement method. First, a measurement sample is prepared by filling a cosmetic material melted by heating into an ointment container having a capacity of 20 mL, and leaving at 25° C. overnight. Hardness of the measurement sample is measured by using a rheometer (manufactured by Rheo-Technology, Ltd.) at 25° C. under conditions of a pressure sensitive axis with a disk having a diameter of 1 mm, a needle penetration rate of 6 cm/min, and a penetration depth of 10 mm.

H1 and H2 may be 2.5 N to 5.0 N and 0.5 N to 3.0 N, and may be 3.5 N to 4.5 N and 1.5 N to 2.5 N, respectively, from a viewpoint of ease of obtaining different makeup effects. H1/H2 may be 3.0/1 to 1.5/1 or 2.5/1 to 2/1 from the same viewpoint.

Regarding the stick-shaped cosmetic material according to the present embodiment, from a viewpoint of achieving different makeup effects, ease of drawing, and prevention of unbalanced core reduction, a content of the component (A1) and a content of the component (A2) in the first cosmetic composition may be respectively 30 mass % to 40 mass % and 0.5 mass % to 10 mass % based on a total amount of the first cosmetic composition, a content of the component (A1) and a content of the component (A2) in the second cosmetic composition may be respectively 20 mass % to 30 mass % and 2 mass % to 15 mass % based on a total amount of the second cosmetic composition, and hardness at room temperature (25° C.) of the first cosmetic composition and the second cosmetic composition may be respectively 2.5 N to 4.5 N and 0.5 N to 2.5 N. Regarding the stick-shaped cosmetic material according to the present embodiment, from a viewpoint of obtaining different feelings of use, ease of drawing, and prevention of unbalanced core reduction, a content of the component (A1) and a content of the component (A2) in the first cosmetic composition may be respectively 30 mass % to 37 mass % and 3 mass % to 7 mass % based on a total amount of the first cosmetic composition, a content of the component (A1) and a content of the component (A2) in the second cosmetic composition may be respectively 20 mass % to 30 mass % and 2 mass % to 13 mass % based on a total amount of the second cosmetic composition, and hardness at room temperature (25° C.) of the first cosmetic composition and the second cosmetic composition may be respectively 2.5 N to 4.5 N and 0.5 N to 2.5 N.

In the stick-shaped cosmetic material according to the present embodiment, the first region may extend in the longitudinal direction of the stick-shaped cosmetic material, and may include two or more layers of the first cosmetic composition. The second region may extend in the longitudinal direction of the stick-shaped cosmetic material, and may include two or more layers of the second cosmetic composition. The stick-shaped cosmetic material according to the present embodiment may include two or more first regions or two or more second regions.

The stick-shaped cosmetic material according to the present embodiment may further include one or more third regions other than the first region and the second region as long as the effect of the present disclosure are not impaired. The third region may contain a cosmetic composition containing the oil component and the powder component described above, and may contain other necessary components. The third region may be provided for reinforcing the stick-shaped cosmetic material or for adjusting the appearance of the stick-shaped cosmetic material. Bulk hardness of the cosmetic composition of the third region may be appropriately set.

The stick-shaped cosmetic material according to the present embodiment may be produced by, for example, the following procedure. First, the first cosmetic composition and the second cosmetic composition are each prepared by mixing the oil component and the powder component described above, and other necessary components. As a preparation method, for example, the oil component and other necessary components are mixed, heated and dissolved, and then the powder component is added to the mixture, and the obtained mixture is uniformly dispersed by a three-roll mill or a stirrer. Next, the stick-shaped cosmetic material may be produced by a method of melting and filling the first cosmetic composition and the second cosmetic composition into a metal mold, a method of extrusion-molding the first cosmetic composition and the second cosmetic composition, or a method of combining the methods.

Examples of the method for melting and filling the first cosmetic composition and the second cosmetic composition into the metal mold include the following method. (i) The melted second cosmetic composition is filled into a metal mold for forming the second region, and is solidified. (ii) A part of the metal mold containing the solidified second cosmetic composition is replaced with a metal mold for forming the first region, and the melted first cosmetic composition is filled into the metal mold and is solidified. (iii) Next, the stick-shaped cosmetic material is taken out from the metal mold. In the above method, the second region is first formed from a viewpoint of ease of filling, but the stick-shaped cosmetic material may be formed in order of the first region and the second region. The cosmetic compositions may be solidified by natural cooling or cooling.

In the case of the extrusion molding, for example, the cosmetic composition (for example, a first cosmetic composition and a second cosmetic composition) for forming each region may be simultaneously extruded and molded in a predetermined shape and integrated.

In the case of the combination of the extrusion molding and the melting and filling, for example, the first cosmetic composition extruded and molded into a shape of the first region may be placed in a metal mold for forming the second region, and the melted second cosmetic composition may be filled in a gap in the metal mold and solidified.

In each of the methods described above, the cosmetic composition may be solidified by natural cooling or cooling. The molded stick-shaped cosmetic material may be thermally treated.

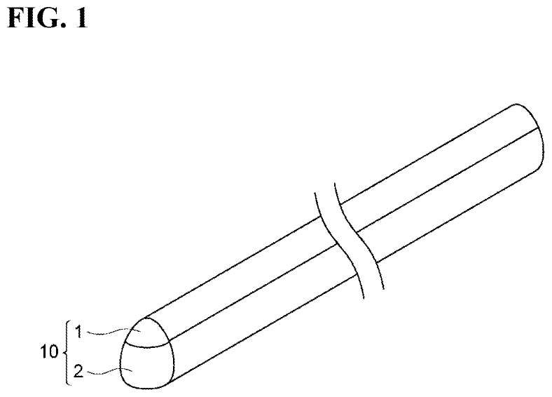

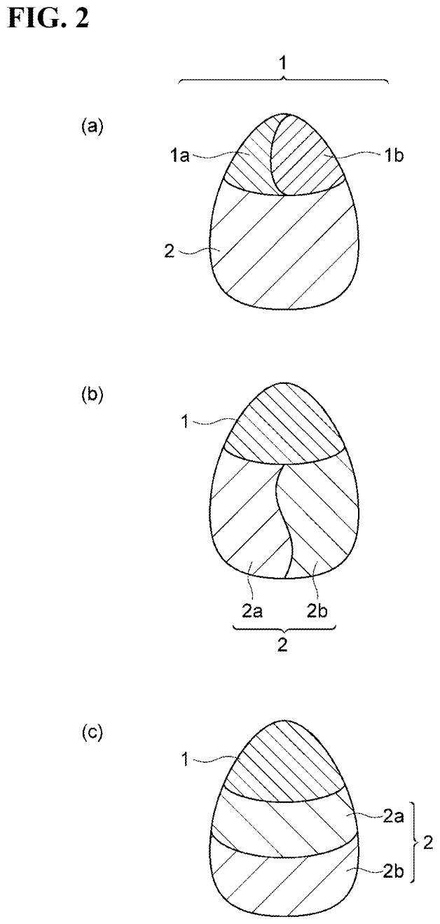

FIG. 1 is a perspective view illustrating an embodiment of the stick-shaped cosmetic material according to the present disclosure. A stick-shaped cosmetic material 10 illustrated in FIG. 1 includes a first region 1 and a second region 2 which are divided in a longitudinal direction of the stick-shaped cosmetic material 10. The first region 1 and the second region 2 are each formed of the first cosmetic composition and the second cosmetic composition described above. In the stick-shaped cosmetic material 10, for example, the first region may be a hard layer and the second region may be a soft layer. This allows for achieving different makeup effects.

(a) to (c) of FIG. 2 illustrate schematic sectional views of other embodiments of the stick-shaped cosmetic material according to the present disclosure, and illustrates cross sections orthogonal to a longitudinal direction of the stick-shaped cosmetic material. The stick-shaped cosmetic material illustrated in (a) of FIG. 2 includes a first region 1 including layers 1a and 1b, and a second region 2. The stick-shaped cosmetic materials illustrated in (b) and (c) of FIG. 2 include a first region 1 and a second region 2 including layers 2a and 2b. The layers 1a and 1b and the layers 2a and 2b may be formed of the first cosmetic composition and the second cosmetic composition described above, respectively. Even in these stick-shaped cosmetic materials, different makeup effects can be achieved by, for example, setting the first region as a hard layer and setting the second region as a soft layer, and the layers 1a and 1b and the layers 2a and 2b may be layers having different colors, for example.

When the first region and/or the second region includes two or more layers having different colors in the stick-shaped cosmetic material according to the present embodiment, from a viewpoint of ease of selectively drawing of each layer, the layers of the first region and/or the layers of the second region may have only one outer surface having a corner at the tip of the stick-shaped cosmetic material, as in the layers 1a and 1b illustrated in (a) of FIG. 2 and the layers 2a and 2b illustrated in (b) of FIG. 2. When the second region includes two or more layers having different colors in the stick-shaped cosmetic material according to the present embodiment, from a viewpoint of ease of achieving a finish having a stereoscopic effect even when the layers of the second region are simultaneously applied, and ease of producing, the second region may include one layer adjacent to the first region and one or more layers not adjacent to the first region as illustrated in (c) of FIG. 2.

The cross section of the stick-shaped cosmetic material 10 is substantially fan-shaped, but the shape of the stick-shaped cosmetic material is not limited. For example, the shape of the cross section orthogonal to the longitudinal direction (a long axis direction) of the stick-shaped cosmetic material may be a circle, an ellipse, a triangle, a rectangle, or a polygon (the number of corners is 5 or more).

When an area of the first region and an area of the second region in the cross section of the stick-shaped cosmetic material according to the present embodiment are defined as S1 and S2, from the viewpoint of selectively drawing with different makeup effects, a relation of S1<S2 may be satisfied, S1/S2 may be 0.5/9.5 to 4.5/5.5, or may be 1.0/9.0 to 4.0/6.0. When the stick-shaped cosmetic material have two or more of the first regions and/or second regions, a total area of the first regions and a total area of the second regions may satisfy the above relations. When the stick-shaped cosmetic material according to the present embodiment includes the third region, an area S3 of the third region in the cross section may be, for example, smaller than S2 or smaller than S1 and S2.

When the stick-shaped cosmetic material according to the present embodiment includes the first region, and the second region including the layer adjacent to the first region and the layer not adjacent to the first region, when the area of the first region in the cross section is defined as S1a and the area of the two layers in the second region are defined as S2a and S2b, respectively (S2b refers to the area of the layer not adjacent to the first region), S2a/S2b may be 1.0/9.0 to 9.0/1.0, or 3.0/7.0 to 5.0/5.0, and S1a/S2a/S2b may be 0.5/1.5/8.0 to 4.5/4.5/1.0. In this case, the second region may include two layers having different colors, and a finish having a stereoscopic effect can be easily achieved even when the layers of the second region are simultaneously applied, and the stick-shaped cosmetic material can be easily produced.

A maximum diameter of the stick-shaped cosmetic material according to the present embodiment may be 1.0 mm or more and 10.0 mm or less, may be 2.0 mm or more and 8.0 mm or less, and may be 3.0 mm or more and 6.0 mm or less.

The stick-shaped cosmetic material according to the present embodiment is preferably used as a cosmetic material for eyebrows having two or more functions among an eyebrow pencil, an eyebrow powder, and a concealer, in particular, having both functions of an eyebrow pencil and an eyebrow powder. The stick-shaped cosmetic material according to the present embodiment can be used in a pencil-shaped cosmetic product used by feeding a stick-shaped cosmetic material.

### EXAMPLES

Hereinafter, the present disclosure will be described in more detail by way of Examples, but the technical scope of the present disclosure is not limited to these Examples. Numerical values in tables each indicate a content (mass %) based on a total amount of a cosmetic base material.

Prior to Examples, an evaluation method adopted in each Example will be described.

### Bulk Hardness of Cosmetic Composition

The bulk hardness of the cosmetic composition was determined by the following measurement method. First, a measurement sample was prepared by filling a cosmetic material melted by heating into an ointment container having a capacity of 20 mL, and leaving at 25° C. overnight. Hardness of the measurement sample was measured by using a rheometer (manufactured by Rheo-Technology, Ltd.) at 25° C. with a pressure sensitive axis with a disk having a diameter of 1 mm, at a needle penetration rate of 6 cm/min, and at a penetration depth of 10 mm.

### Evaluation of Ease of Drawing

With respect to stick-shaped cosmetic materials of Examples and Comparative Examples, five cosmetic evaluation professional panelists evaluated whether they felt no catching and whether a uniform drawn line was drawn when a first region and a second region were applied to a skin together. In the evaluation of ease of drawing, five-grade evaluation was performed according to the following evaluation criteria, a score was given to each sample, and a total score of all panelists was determined according to the following criteria.
[Score: Evaluation Criteria] 5 points: very good; 4 points: good; 3 points: normal; 2 points: slightly poor; 1 point: poor
[Determination Criteria (total point of score)] A: 20 or more; B: 15 or more and less than 20; C 10 or more and less than 15; D: less than 10

### Evaluation of Selectively Drawing in Terms of Makeup Effects

With respect to the stick-shaped cosmetic materials of Examples and Comparative Examples, five cosmetic evaluation professional panelists evaluated whether different makeup effects (the first layer: a clear drawn line, the second layer: a feathered drawn line) were felt when the first layer and the second layer were separately applied to the skin by adjusting an angle of each cosmetic material to be applied to the skin. In the evaluation of selectively drawing in terms of makeup effects, five-grade evaluation was performed according to the following evaluation criteria, a score was given to each sample, and a total score of all panelists was determined according to the following criteria.
[Score: Evaluation Criteria] 5 points: very good; 4 points: good; 3 points: normal; 2 points: slightly poor; 1 point: poor
[Determination Criteria (total point of score)] A: 20 or more; B: 15 or more and less than 20; C 10 or more and less than 15; D: less than 10

### Evaluation of Unbalanced Reduction

A writing test was performed in which a line having a length of 10 cm was drawn ten times on a surface of a bioskin (manufactured by Beaulax Co., Ltd.)) at a feeding core length of 2 mm, and the degree of reduction of the core was evaluated according to the following evaluation criteria. [Evaluation Criteria]: A: The reduction of the two layers is equivalent and no chipping occurs; B: A slightly difference in the reduction of the two layers, but no chipping occurs; C: A difference in the reduction of the two layers; D: A significant difference in the reduction of the two layers

### Examples 1 to 13 and Comparative Examples 1 to 4

In each of Examples 1 to 13 and Comparative Examples 1 to 4, a first cosmetic composition having a composition (mass %) of a first layer shown in Tables 1 to 4 and a second cosmetic composition having a composition (mass %) of a second layer shown in Tables 1 to 4 were prepared by the following production method 1.

### Production Method 1

A powder component was mixed into a mixture obtained by heating and dissolving an oil component at 95° C. to 105° C., and the mixture was uniformly dispersed by using three rolls.

Next, the first cosmetic composition and the second cosmetic composition were used to produce, by the following production method 2, a stick-shaped cosmetic material having the same shape as the stick-shaped cosmetic material illustrated in FIG. 1 (that is, having a substantially fan-shaped cross section) and having a maximum diameter of 3.5 mm, and in which a ratio S1/S2 of a cross-sectional area S1 of the first layer and a cross-sectional area S2 of the second layer was 2.2/7.8.

### Production Method 2

(i) The melted second cosmetic composition was filled into a metal mold for forming the second layer, and the filled composition was solidified. (ii) A part of the metal mold containing the second layer was replaced with a metal mold for forming the first layer, and the melted first cosmetic composition was filled into the metal mold and was solidified. (iii) The stick-shaped cosmetic material was taken out from the metal mold after confirming that the first layer was solidified.

The cosmetic compositions and the stick-shaped cosmetic materials obtained above were evaluated as described above. Results are illustrated in Tables 1 to 4.

TABLE 1

| | | | Example 1 | | Example 2 | | Example 3 | | Example 4 | | Example 5 | |
|---|---|---|---|---|---|---|---|---|---|---|---|---|
| | | | First | Second | First | Second | First | Second | First | Second | First | Second |
| | | | layer | layer | layer | layer | layer | layer | layer | layer | layer | layer |
| Oil | (A1) | Synthetic wax | 12 | 8 | 12 | 8 | 12 | 8 | 12 | 8 | 12 | 8 |
| component | | Polyethylene | 5 | 8 | 5 | 8 | 5 | 8 | 5 | 8 | 5 | 8 |
| (A) | | wax | | | | | | | | | | |
| | | Ceresin | 8 | 10 | 8 | 10 | 8 | 10 | 8 | 10 | 8 | 10 |
| | | Hydrogenated | 10 | — | 10 | — | 10 | — | 10 | — | 10 | — |
| | | castor oil | | | | | | | | | | |
| | (A2) | Fatty acid | — | 18 | — | 15 | — | 13 | — | 10 | — | 7 |
| | | triglyceride-1 | | | | | | | | | | |
| | | Fatty acid | 5 | — | 5 | — | 5 | — | 5 | — | 5 | — |
| | | triglyceride-2 | | | | | | | | | | |
| | (A3) | Caprylic/capric | 12 | 15 | 12 | 15 | 12 | 15 | 12 | 15 | 12 | 15 |
| | | triglyceride | | | | | | | | | | |
| | | Triethylhexanoin | 5 | — | 5 | — | 5 | — | 5 | — | 5 | — |
| | | Neopentyl glycol | — | — | — | 3 | — | 5 | — | 8 | — | 11 |
| | | diethylhexanoate | | | | | | | | | | |
| | Heavy liquid | | 4 | — | 4 | — | 4 | — | 4 | — | 4 | — |
| | isoparaffin | | | | | | | | | | | |
| | Diisostearyl malate | | 3 | — | 3 | — | 3 | — | 3 | — | 3 | — |
| | Sorbitan | | 1 | 1 | 1 | 1 | 1 | 1 | 1 | 1 | 1 | 1 |
| | sesquiisostearate | | | | | | | | | | | |
| Powder | (B1) | Red iron oxide | 3.3 | 3.3 | 3.3 | 3.3 | 3.3 | 3.3 | 3.3 | 3.3 | 3.3 | 3.3 |
| component | | Yellow iron | 10.0 | 9.0 | 10.0 | 9.0 | 10.0 | 9.0 | 10.0 | 9.0 | 10.0 | 9.0 |
| (B) | | oxide | | | | | | | | | | |
| | | Black iron oxide | 4.1 | 1.7 | 4.1 | 1.7 | 4.1 | 1.7 | 4.1 | 1.7 | 4.1 | 1.7 |
| | | Titanium oxide | 12.0 | 7.0 | 12.0 | 7.0 | 12.0 | 7.0 | 12.0 | 7.0 | 12.0 | 7.0 |
| | (B2) | Mica | 5.6 | 19 | 5.6 | 19 | 5.6 | 19 | 5.6 | 19 | 5.6 | 19 |

| Evaluation | Bulk hardness | 4.23 | 2.53 | 4.23 | 2.23 | 4.23 | 2.00 | 4.23 | 2.00 | 4.23 | 1.97 |
|---|---|---|---|---|---|---|---|---|---|---|---|
| | (N) | | | | | | | | | | |
| | Ease of drawing | C | | C | | B | | A | | A | |
| | Selectively | B | | B | | A | | A | | A | |
| | drawing in terms | | | | | | | | | | |
| | of makeup | | | | | | | | | | |
| | effects | | | | | | | | | | |
| | Unbalanced | A | | A | | A | | A | | A | |
| | reduction | | | | | | | | | | |

TABLE 2

| | | | Example 6 | | Example 7 | | Example 8 | | Example 9 | | Example 10 | |
|---|---|---|---|---|---|---|---|---|---|---|---|---|
| | | | First | Second | First | Second | First | Second | First | Second | First | Second |
| | | | layer | layer | layer | layer | layer | layer | layer | layer | layer | layer |
| Oil | (A1) | Synthetic wax | 12 | 8 | 12 | 8 | 12 | 8 | 12 | 8 | 12 | 8 |
| component | | Polyethylene | 5 | 8 | 5 | 8 | 5 | 8 | 5 | 8 | — | — |
| (A) | | wax | | | | | | | | | | |
| | | Microcrystalline | — | — | — | — | — | — | — | — | 5 | 8 |
| | | wax | | | | | | | | | | |
| | | Ceresin | 8 | 10 | 8 | 10 | 8 | 10 | 8 | 10 | 8 | 10 |
| | | Hydrogenated | 10 | — | 10 | — | 10 | — | 10 | — | 10 | — |
| | | castor oil | | | | | | | | | | |
| | (A2) | Fatty acid | — | 5 | — | 2 | — | 0.5 | — | — | — | 1 |
| | | triglyceride-1 | | | | | | | | | | |
| | | Fatty acid | 5 | — | 5 | — | 5 | — | 5 | — | — | — |
| | | triglyceride-2 | | | | | | | | | | |
| | | Fatty acid | — | — | — | — | — | — | — | — | 5 | 10 |
| | | triglyceride-3 | | | | | | | | | | |
| | | Fatty acid | — | — | — | — | — | — | — | 10 | — | — |
| | | triglyceride-4 | | | | | | | | | | |
| | (A3) | Caprylic/capric | 12 | 15 | 12 | 15 | 12 | 15 | 12 | 15 | 12 | 15 |
| | | triglyceride | | | | | | | | | | |
| | | Triethylhexanoin | 5 | — | 5 | — | 5 | — | 5 | — | 5 | — |
| | | Neopentyl glycol | — | 13 | — | 16 | — | 17.5 | — | 8 | — | 8 |
| | | diethylhexanoate | | | | | | | | | | |
| | Heavy liquid | | 4 | — | 4 | — | 4 | — | 4 | — | 4 | — |
| | isoparaffin | | | | | | | | | | | |
| | Diisostearyl malate | | 3 | — | 3 | — | 3 | — | 3 | — | 3 | — |
| | Sorbitan | | 1 | 1 | 1 | 1 | 1 | 1 | 1 | 1 | 1 | 1 |
| | sesquiisostearate | | | | | | | | | | | |
| Powder | (B1) | Red iron oxide | 3.3 | 3.3 | 3.3 | 3.3 | 3.3 | 3.3 | 3.3 | 3.3 | 3.3 | 3.3 |
| component | | Yellow iron | 10.0 | 9.0 | 10.0 | 9.0 | 10.0 | 9.0 | 10.0 | 9.0 | 10.0 | 9.0 |
| (B) | | oxide | | | | | | | | | | |
| | | Black iron oxide | 4.1 | 1.7 | 4.1 | 1.7 | 4.1 | 1.7 | 4.1 | 1.7 | 4.1 | 1.7 |
| | | Titanium oxide | 12.0 | 7.0 | 12.0 | 7.0 | 12.0 | 7.0 | 12.0 | 7.0 | 12.0 | 7.0 |
| | (B2) | Mica | 5.6 | 19 | 5.6 | 19 | 5.6 | 19 | 5.6 | 19 | 5.3 | 19 |

| Evaluation | Bulk hardness | 4.23 | 1.87 | 4.23 | 1.9 | 4.23 | 1.97 | 4.23 | 4.57 | 3.57 | 1.77 |
|---|---|---|---|---|---|---|---|---|---|---|---|
| | (N) | | | | | | | | | | |
| | Ease of drawing | A | | A | | B | | B | | A | |
| | Selectively | A | | B | | C | | C | | A | |
| | drawing in terms | | | | | | | | | | |
| | of makeup | | | | | | | | | | |
| | effects | | | | | | | | | | |
| | Unbalanced | B | | B | | B | | A | | A | |
| | reduction | | | | | | | | | | |

TABLE 3

| | | | Example 11 | | Example 12 | | Example 13 | |
|---|---|---|---|---|---|---|---|---|
| | | | First | Second | First | Second | First | Second |
| | | | layer | layer | layer | layer | layer | layer |
| Oil | (A1) | Synthetic wax | 8 | 8 | 16 | 8 | 12 | 8 |
| component | | Polyethylene wax | 5 | 8 | 5 | 8 | 5 | 8 |
| (A) | | Ceresin | 8 | 10 | 8 | 10 | 8 | 10 |
| | | Hydrogenated castor | 10 | — | 10 | — | 10 | — |
| | | oil | | | | | | |
| | (A2) | Fatty acid | — | 7 | — | 7 | — | 7 |
| | | triglyceride-1 | | | | | | |
| | | Fatty acid | 9 | — | 1 | — | — | — |
| | | triglyceride-2 | | | | | | |
| | (A3) | Caprylic/capric | 12 | 15 | 12 | 15 | 17 | 15 |
| | | triglyceride | | | | | | |
| | | Triethylhexanoin | 5 | — | 5 | — | 5 | — |
| | | Neopentyl glycol | — | 11 | — | 11 | — | 11 |
| | | diethylhexanoate | | | | | | |
| | Heavy liquid isoparaffin | | 4 | — | 4 | — | 4 | — |
| | Diisostearyl malate | | 3 | — | 3 | — | 3 | — |
| | Sorbitan sesquiisostearate | | 1 | 1 | 1 | 1 | 1 | 1 |
| Powder | (B1) | Red iron oxide | 3.3 | 3.3 | 3.3 | 3.3 | 3.3 | 3.3 |
| component | | Yellow iron oxide | 10.0 | 9.0 | 10.0 | 9.0 | 10.0 | 9.0 |
| (B) | | Black iron oxide | 4.1 | 1.7 | 4.1 | 1.7 | 4.1 | 1.7 |
| | | Titanium oxide | 12.0 | 7.0 | 12.0 | 7.0 | 12.0 | 7.0 |
| | (B2) | Mica | 5.6 | 19 | 5.6 | 19 | 5.6 | 19 |

| Evaluation | Bulk hardness (N) | 2.93 | 1.97 | 5.33 | 1.97 | 3.73 | 1.97 |
|---|---|---|---|---|---|---|---|
| | Ease of drawing | A | | C | | B | |
| | Selectively drawing | A | | B | | B | |
| | in terms of makeup | | | | | | |
| | effects | | | | | | |
| | Unbalanced | A | | C | | B | |
| | reduction | | | | | | |

TABLE 4

| | | | Comparative | | Comparative | | Comparative | | Comparative | |
|---|---|---|---|---|---|---|---|---|---|---|
| | | | Example 1 | | Example 2 | | Example 3 | | Example 4 | |
| | | | First | Second | First | Second | First | Second | First | Second |
| | | | layer | layer | layer | layer | layer | layer | layer | layer |
| Oil | (A1) | Synthetic wax | 12 | 8 | 12 | 8 | 12 | 8 | 12 | 8 |
| component | | Polyethylene | 5 | 8 | 5 | 8 | 5 | 8 | 5 | 8 |
| (A) | | wax | | | | | | | | |
| | | Ceresin | 8 | 10 | 8 | 10 | 8 | 10 | 8 | 10 |
| | | Hydrogenated | 10 | — | 10 | — | 10 | — | 10 | — |
| | | castor oil | | | | | | | | |
| | (A2) | Fatty acid | 5 | — | 5 | — | — | — | — | — |
| | | triglyceride-2 | | | | | | | | |
| | (A3) | Caprylic/capric | 12 | 25 | 12 | 15 | 17 | 25 | 12 | 15 |
| | | triglyceride | | | | | | | | |
| | | Triethylhexanoin | 5 | — | 5 | — | 5 | — | 5 | — |
| | | Neopentyl glycol | — | 8 | — | 8 | — | 8 | — | 8 |
| | | diethylhexanoate | | | | | | | | |
| | Vaseline | | — | — | — | 10 | — | — | 5 | 10 |
| | Heavy liquid | | 4 | — | 4 | — | 4 | — | 4 | — |
| | isoparaffin | | | | | | | | | |
| | Diisostearyl malate | | 3 | — | 3 | — | 3 | — | 3 | — |
| | Sorbitan | | 1 | 1 | 1 | 1 | 1 | 1 | 1 | 1 |
| | sesquiisostearate | | | | | | | | | |
| Powder | (B1) | Red iron oxide | 3.3 | 3.3 | 3.3 | 3.3 | 3.3 | 3.3 | 3.3 | 3.3 |
| component | | Yellow iron | 10.0 | 9.0 | 10.0 | 9.0 | 10.0 | 9.0 | 10.0 | 9.0 |
| (B) | | oxide | | | | | | | | |
| | | Black iron oxide | 4.1 | 1.7 | 4.1 | 1.7 | 4.1 | 1.7 | 4.1 | 1.7 |
| | | Titanium oxide | 12.0 | 7.0 | 12.0 | 7.0 | 12.0 | 7.0 | 12.0 | 7.0 |
| | (B2) | Mica | 5.6 | 19 | 5.6 | 19 | 5.6 | 19 | 5.6 | 19 |

| Evaluation | Bulk hardness | 4.23 | 2.17 | 4.23 | 2.17 | 3.73 | 2.17 | 3.67 | 2.17 |
|---|---|---|---|---|---|---|---|---|---|
| | (N) | | | | | | | | |
| | Ease of drawing | D | | D | | A | | D | |
| | Selectively | D | | D | | D | | D | |
| | drawing in terms | | | | | | | | |
| | of makeup | | | | | | | | |
| | effects | | | | | | | | |
| | Unbalanced | C | | D | | B | | C | |
| | reduction | | | | | | | | |

In Tables 1 to 4 and Table 5 to be described later, details of each raw material are as follows. synthetic wax: melting point: 83.8° C.; polyethylene wax: melting point: 89.0° C.; microcrystalline wax: melting point: 73.2° C.; ceresin: melting point: 67.0° C.; hydrogenated castor oil: melting point: 86.0° C.; fatty acid triglyceride-1: “Lipocire A SG” (C10-18 triglycerides, manufactured by GATTEFOSSE SAS, trade name): melting point: 31.2° C.; fatty acid triglyceride-2: “Lipocire DM SG” (C10-18 triglycerides, manufactured by GATTEFOSSE SAS, trade name): melting point: 38.1° C.; fatty acid triglyceride-3: “salacos 334” (caprylic/capric/myristic/stearic triglyceride, manufactured by Nisshin OilliO Group, Ltd., trade name): melting point: 40.0° C.; fatty acid triglyceride-4: “SYNCROWAX HRC” (glyceryl tribehenate, manufactured by Croda Japan K.K., trade name): melting point: 58.1° C.; caprylic/capric triglyceride: viscosity: 26 mPa·s at 25° C.; triethylhexanoin: viscosity: 31 mPa·s at 25° C.; neopentyl glycol diethylhexanoate: viscosity: 12 mPa·s at 25° C.

The above melting point was measured according to the following method. About 5 mg of a sample was weighed and placed in an aluminum sample pan. An aluminum cover was attached to the pan, and the pan was installed in a differential scanning calorimeter “DSC7020” (manufactured by Hitachi High-Tech Science Corporation, product name). By using an electric cooling unit “Polyscience” (manufactured by Hitachi High-Tech Science Corporation, product name), a sample and a reference sample were held at −10° C. for 1 min under a nitrogen gas flow rate of 30 mL/min to 35 mL/min, and then heated from 0° C. to 120° C. with a heating rate of 10° C./min, cooled from 120° C. to −10° C. with a cooling condition of −10° C./min, held at −10° C. for 5 min, and then heated again from −10° C. to 120° C. with a heating rate of 10° C./min, to determine a melting endothermic curve. A peak temperature of the melting endothermic curve during the second heating at this time was determined as the melting point. If the melting endothermic curve had two or more peak temperatures, a peak temperature having a highest melting temperature was determined as the melting point.

The viscosity at 25° C. of a liquid oil was obtained by measuring a sample at 25° C. by using a B-type viscometer with a BL adapter and at a rotation speed of 12 rpm.

### Example 14

A first cosmetic composition was prepared in the same manner as in Example 4. A second cosmetic composition having a composition (mass %) of the following second layer was prepared by the above production method 1.

### Composition of Second Layer

(Components)
(Blending Ratios (mass %))

| | | |
|---|---|---|
| 1. | synthetic wax (melting point: 96.7° C.) | 8 |
| 2. | dextrin palmitate | 5 |
| 3. | fatty acid triglyceride-1 *1 | 10 |
| 4. | caprylic/capric triglyceride *2 | 10 |
| 5. | polyglyceryl triisostearate-2 | 12.6 |
| 6. | diisostearyl malate | 14.2 |
| 7. | acrylates/stearyl acrylate/dimethicone | 20 |
| | methacrylate copolymer | |
| 8. | sorbitan sesquiisostearate | 1 |
| 9. | titanium oxide | 7.2 |
| 10. | red iron oxide | 0.4 |
| 11. | black iron oxide | 0.1 |
| 12. | yellow iron oxide | 1.5 |
| 13. | mica | 10 |

*1: The same as described above.
*2: viscosity: 26 mPa · s at 25° C.

Next, the first cosmetic composition and the second cosmetic composition were used to produce, by the above production method 2, a stick-shaped cosmetic material having the same shape as the stick-shaped cosmetic material illustrated in FIG. 1 (that is, having a substantially fan-shaped cross section) and having a maximum diameter of 3.5 mm, and in which a ratio S1/S2 of a cross-sectional area S1 of a first layer and a cross-sectional area S2 of the second layer was 4.5/5.5.

When the above evaluations were performed on the second cosmetic composition and the stick-shaped cosmetic material obtained as described above, bulk hardness of the second cosmetic composition was 0.8 N, “ease of application”, “selectively drawing in terms of makeup effects”, and “unbalanced reduction” were all evaluated as “B”.

### Example 15

A first cosmetic composition was prepared in the same manner as in Example 4. A second cosmetic composition having a composition (mass %) of the following second layer was prepared by the above production method 1.

### Composition of Second Layer

(Components)
(Blending Ratios (mass %))

| | | |
|---|---|---|
| 1. | synthetic wax (melting point: 96.7° C.) | 4 |
| 2. | candelilla wax (melting point: 70° C.) | 4.5 |
| 3. | carnauba wax (melting point: 83° C.) | 1 |
| 4. | fatty acid triglyceride-1 *3 | 10 |
| 5. | polyglyceryl triisostearate-2 | 18 |
| 6. | dipentaerythrityl hexahydroxystearate/ | 4 |
| | hexastearate/hexarosinate | |
| 7. | ethylhexyl stearate*4 | 15 |
| 8. | sorbitan sesquioleate | 1 |
| 9. | titanium oxide | 25 |
| 10. | red iron oxide | 2.1 |
| 11. | black iron oxide | 0.1 |
| 12. | yellow iron oxide | 5.3 |
| 13. | mica | 10 |

*3: The same as described above.
*4viscosity: 15 mPa · s at 25° C.

Next, the first cosmetic composition and the second cosmetic composition were used to produce, by the above production method 2, a stick-shaped cosmetic material having the same shape as the stick-shaped cosmetic material illustrated in FIG. 1 (that is, having a substantially fan-shaped cross section) and having a maximum diameter of 3.5 mm, and in which a ratio S1/S2 of a cross-sectional area S1 of a first layer and a cross-sectional area S2 of the second layer was 2.2/7.8.

When the above evaluations were performed on the second cosmetic composition and the stick-shaped cosmetic material obtained as described above, bulk hardness of the second cosmetic composition was 1.1 N, “ease of application”, “selectively drawing in terms of makeup effects”, and “unbalanced reduction” were all evaluated as “B”.

### Examples 16 and 17

In each of Examples 16 and 17, a first cosmetic composition having a composition (mass %) of a first layer shown in Table 5, a second cosmetic composition a having a composition (mass %) of a layer 2a shown in Table 5, and a second cosmetic composition b having a composition (mass %) of a layer 2b shown in Table 5 were prepared in the same manner as in the production method 1.

Next, the first cosmetic composition, the second cosmetic composition a, and the second cosmetic composition b were used to produce, by the following production method 3, a stick-shaped cosmetic material having the same shape as the stick-shaped cosmetic material illustrated in (c) of FIG. 2 (that is, having a substantially fan-shaped cross section) and having a maximum diameter of 4.0 mm, and in which a ratio S1/S2a/S2b of a cross-sectional area S1 of the first layer, a cross-sectional area S2a of the layer 2a, and a cross-sectional area S2b of the layer 2b was 2/3/5.

### Production Method 3

(i) Metal molds for forming the first layer and the layer 2b were respectively filled with the melted first cosmetic composition and second cosmetic composition b, with the metal molds set in a state in which a mold corresponding to the layer 2a was fitted. (ii) After confirming that the first layer and the layer 2b were solidified, the mold corresponding to the layer 2a was removed, and then the molten second cosmetic composition a was filled in a vacant portion. (iii) The stick-shaped cosmetic material was taken out from the metal mold after confirming that the layer 2a was solidified.

The cosmetic compositions and the stick-shaped cosmetic materials obtained above were evaluated as described above. Results are illustrated in Table 5.

TABLE 5

| | | | Example 16 | | | Example 17 | | |
|---|---|---|---|---|---|---|---|---|
| | | | First | | | First | | |
| | | | layer | Layer 2a | Layer 2b | layer | Layer 2a | Layer 2b |
| Oil | (A1) | Synthetic wax | 12 | 8 | 8 | 12 | 8 | 8 |
| component | | Polyethylene wax | 5 | 8 | 8 | 5 | 8 | 8 |
| (A) | | Microcrystalline wax | — | — | — | — | — | — |
| | | Ceresin | 8 | 10 | 10 | 8 | 10 | 10 |
| | | Hydrogenated castor oil | 10 | — | — | 10 | — | — |
| | (A2) | Fatty acid triglyceride-1 | — | 10 | 10 | — | 7 | 7 |
| | | Fatty acid triglyceride-2 | 5 | — | — | 5 | — | — |
| | | Fatty acid triglyceride-3 | — | — | — | — | — | — |
| | | Fatty acid triglyceride-4 | — | — | — | — | — | — |
| | (A3) | Caprylic/capric triglyceride | 12 | 15 | 15 | 12 | 15 | 15 |
| | | Triethylhexanoin | 5 | — | — | 5 | — | — |
| | | Neopentyl glycol | — | 8 | 8 | — | 11 | 11 |
| | | diethylhexanoate | | | | | | |
| | Heavy liquid isoparaffin | | 4 | — | — | 4 | — | — |
| | Diisostearyl malate | | 3 | — | — | 3 | — | — |
| | Sorbitan sesquiisostearate | | 1 | 1 | 1 | 1 | 1 | 1 |
| Powder | (B1) | Red iron oxide | 3.3 | 3.3 | 2.6 | 3.3 | 3.3 | 2.6 |
| component | | Yellow iron oxide | 10.0 | 9.0 | 7.2 | 10.0 | 9.0 | 7.2 |
| (B) | | Black iron oxide | 4.1 | 1.7 | 1.3 | 4.1 | 1.7 | 1.3 |
| | | Titanium oxide | 12.0 | 7.0 | 9.9 | 12.0 | 7.0 | 9.9 |
| | (B2) | Mica | 5.6 | 19 | 19 | 5.6 | 19 | 19 |

| Evaluation | Bulk hardness (N) | 4.23 | 2.00 | 2.00 | 4.23 | 1.97 | 1.97 |
|---|---|---|---|---|---|---|---|
| | Ease of drawing | A | | | A | | |
| | Selectively drawing in terms of | A | | | A | | |
| | makeup effects | | | | | | |

| | | |
|---|---|---|
| Unbalanced reduction | A | A |

### Reference Signs List

1: first region

1a, 1b: layer constituting first region

2: second region

2a, 2b: layer constituting second region

10: stick-shaped cosmetic material

1. A stick-shaped cosmetic material comprising two or more regions divided in a longitudinal direction of the stick-shaped cosmetic material,
wherein the two or more regions comprise:
a first region comprising a first cosmetic composition containing a wax (A1) having a melting point of 65° C. or higher as an oil component in the first cosmetic composition, and
a second region comprising a second cosmetic composition containing, as oil components in the second cosmetic composition, the wax (A1) having a melting point of 65° C. or higher and a fatty acid triglyceride (A2) having a melting point of 30° C. or higher,
wherein the first region and the second region are adjacent to each other, and
wherein, when a content ratio (mass %) of the wax (A1) based on a total amount of the oil components in the first cosmetic composition and a content ratio (mass %) of the wax (A1) based on a total amount of the oil components in the second cosmetic composition are defined as M1 and M2, respectively, M1>M2 is satisfied.

2. The stick-shaped cosmetic material according to claim 1, wherein, when hardness (N) at 25° C. of the first cosmetic composition and hardness (N) at 25° C. of the second cosmetic composition are defined as H1 and H2, respectively, H1>H2 is satisfied.

3. The stick-shaped cosmetic material according to claim 1, wherein the first cosmetic composition comprises hydrogenated castor oil as the wax (A1).

4. The stick-shaped cosmetic material according to claim 1, wherein the first cosmetic composition comprises the fatty acid triglyceride (A2) having a melting point of 30° C. or higher.

5. The stick-shaped cosmetic material according to claim 4,
wherein a content of the wax (A1) and a content of the fatty acid triglyceride (A2) in the first cosmetic composition are respectively 30 mass % to 37 mass % and 3 mass % to 7 mass % based on the amount of the first cosmetic composition,
wherein a content of the wax (A1) and a content of the fatty acid triglyceride (A2) in the second cosmetic composition are respectively 20 mass % to 30 mass % and 2 mass % to 13 mass % based on the amount of the second cosmetic composition, and
wherein hardness at 25° C. of the first cosmetic composition and hardness at 25° C. of the second cosmetic composition are respectively 2.5 N to 4.5 N and 0.5 N to 2.5 N.

6. The stick-shaped cosmetic material according to claim 1, wherein the fatty acid triglyceride (A2) has a melting point of 60° C. or lower.

7. The stick-shaped cosmetic material according to claim 4, wherein the fatty acid triglyceride (A2) has a melting point of 60° C. or lower.

8. The stick-shaped cosmetic material according to claim 1, wherein the fatty acid triglyceride (A2) has a melting point of 45° C. or lower.

9. The stick-shaped cosmetic material according to claim 4, wherein the fatty acid triglyceride (A2) has a melting point of 45° C. or lower.

10. The stick-shaped cosmetic material according to claim 1, wherein a hydrocarbon group derived from a fatty acid in the fatty acid triglyceride (A2) has 6 to 20 carbon atoms.

11. The stick-shaped cosmetic material according to claim 4, wherein a hydrocarbon group derived from a fatty acid in the fatty acid triglyceride (A2) has 6 to 20 carbon atoms.

12. The stick-shaped cosmetic material according to claim 1, wherein a hydrocarbon group derived from a fatty acid in the fatty acid triglyceride (A2) has 8 to 18 carbon atoms.

13. The stick-shaped cosmetic material according to claim 4, wherein a hydrocarbon group derived from a fatty acid in the fatty acid triglyceride (A2) has 8 to 18 carbon atoms.

14. The stick-shaped cosmetic material according to claim 1, wherein the fatty acid triglyceride (A2) is at least one selected from caprylic/capric/myristic/stearic triglyceride, C10-18 triglycerides, and glyceryl trilaurate.

15. The stick-shaped cosmetic material according to claim 4, wherein the fatty acid triglyceride (A2) is at least one selected from caprylic/capric/myristic/stearic triglyceride, C10-18 triglycerides, and glyceryl trilaurate.

16. The stick-shaped cosmetic material according to claim 1, wherein, when an area of the first region and an area of the second region in the cross section of the stick-shaped cosmetic material are defined as S1 and S2, S1/S2 is 0.5/9.5 to 4.5/5.5.

17. The stick-shaped cosmetic material according to claim 4, wherein, when an area of the first region and an area of the second region in the cross section of the stick-shaped cosmetic material are defined as S1 and S2, S1/S2 is 0.5/9.5 to 4.5/5.5.

18. The stick-shaped cosmetic material according to claim 1, wherein, when an area of the first region and an area of the second region in the cross section of the stick-shaped cosmetic material are defined as S1 and S2, S1/S2 is 1.0/9.0 to 4.0/6.0.

19. The stick-shaped cosmetic material according to claim 4, wherein, when an area of the first region and an area of the second region in the cross section of the stick-shaped cosmetic material are defined as S1 and S2, S1/S2 is 1.0/9.0 to 4.0/6.0.

20. The stick-shaped cosmetic material according to claim 1, the second region includes one layer adjacent to the first region and one or more layers not adjacent to the first region.
